(19) United States
(12) Patent Application Publication
(10) Pub. No.: US 2015/0245769 A1
(43) Pub. Date: Sep. 3, 2015

(54) Optical Measurement Device And Probe System

(71) Applicant: KONICA MINOLTA, INC., Chiyoda-ku, Tokyo (JP)

(72) Inventors: Yusuke Mimura, Chiyoda-ku (JP); Yasuyuki Natsuno, Chiyoda-ku (JP)

(21) Appl. No.: 14/430,125

(22) PCT Filed: Sep. 19, 2013
(86) PCT No.: PCT/JP2013/005537

§ 371 Date: Mar. 20, 2015

(30) Foreign Application Priority Data
Sep. 24, 2012 (JP) 2012-209849

(51) Int. Cl. A61B 5/00; G01J 3/02; A61B 1/07; A61B 1/00; A61B 1/06; G01J 3/44; A61B 1/045

(52) U.S. Cl. CPC A61B 5/0084; G01J 3/4406; G01J 3/0218; A61B 5/0075; A61B 1/045; A61B 1/00009; A61B 1/00006; A61B 1/0661; A61B 1/00096; A61B 1/07; A61B 5/7282; A61B 2562/0233

(57) ABSTRACT

An optical measurement device provided with: a first adjustment optical device for collecting radiation light received by a probe that emits measurement light to a measurement target and receives radiation light radiated from the measurement target, and for emitting the radiation light toward the spectroscope for dividing the radiation light; a detection section for detecting a light intensity distribution of the radiation light; a movement part for moving the first adjustment optical device in a light axis direction of the radiation light and on a plane perpendicular to the light axis direction of the radiation light; and a control section for controlling the movement part. The first adjustment optical device is moved in the light axis direction of the radiation light and on the plane on a basis of a detection result of the detection section such that a reception amount of the radiation light increases.

### TECHNICAL FIELD

The present invention relates to an optical measurement device and a probe system, and in particular, is suitable for a probe system which emits measurement light to a measurement target part of a body lumen (hereinafter simply referred to as “lumen”) and acquires radiation light from the measurement target part to examine for a lesion such as cancer, and its progression.

### BACKGROUND ART

In recent years, a method for observing a lumen using an electron endoscope has been widely accepted. In such an observation method, advantageously, removal of a lesion is not required since a body tissue is directly observed, and therefore burden of an examinee is small. Recently, ultrasound apparatuses and diagnosis apparatuses utilizing various optical principles other than so-called video scopes have been proposed, and some of such apparatuses have been practically used. In this manner, new measurement principles have been introduced, and different measurement principles have been combined.

In addition, it is known that information which cannot be obtained by simply visually recognizing a body tissue image can be obtained by observing and measuring fluorescence from a body tissue and fluorescence from a fluorescence material applied or injected to a body tissue. Such a method is applied in a fluorescence image endoscope system which acquires a fluorescence image using the method and displays the image with a normal visible image in an overlapping manner. Such a system leads to early detection of malignant tumor, and is therefore highly expected.

A method is also known in which, without forming a fluorescence image, a state of a body tissue is determined by acquiring intensity information of fluorescence. In such a method, typically, fluorescence is acquired without using an imaging device mounted in an electron endoscope.

Examples of probes (diagnostic tools) for a fluorescence diagnosis include a probe that is advanced in the body via an endoscope forceps channel, a probe that is integrated into an endoscope, and the like.

Such a probe is connected with a spectroscope and a measurement device having a light source, and is configured to propagate excitation light emitted from the light source and to emit the light to a body tissue as measurement light. The body tissue on which measurement light is applied radiates reflection light including fluorescence as radiation light. After the probe has received the radiation light, the intensity of each wavelength component of the radiation light is measured in the measurement device, thereby examining for a lesion such as cancer and its progression.

However, the amount of light (fluorescence) radiated from a body tissue is extremely small, and therefore it is necessary to increase the amount of the radiation light received by the measurement device as much as possible in order that diagnosis is correctly performed on the basis of the radiation light.

PTL 1 discloses an endoscope apparatus in which an maximum amount of incident light can be always obtained with a maximum efficiency in various light guides. Such an endoscope apparatus is provided with a light amount measurement member having a light reception part at an incident end surface of the light guide, and the relative position of the light collection position of emission light (illumination light) and the incident end surface of the light guide is adjusted on the basis of the output of the light amount measurement member.

PTL 2 discloses a light source apparatus in which an adapter having a detachable light amount measurement member is inserted between the light source of an endoscope apparatus not having the light amount measurement member disclosed in PTL 1 and a light guide, and the position for collecting illuminating light from the light source is adjusted on the basis of measurement results of the light amount measurement member.

### CITATION LIST

### Patent Literature

PTL 1

Japanese Patent Application Laid-Open No. 4-70710

PTL 2

Japanese Patent Application Laid-Open No. 7-181399

### SUMMARY OF INVENTION

### Technical Problem

However, an object of the techniques disclosed in PTLS 1 and 2 is to maximize the amount of light that impinges on the light guide that guides illumination light from the light source in the endoscope. In addition, while PTLS 1 and 2 disclose that a target sample on which illumination light is applied is directly observed from an ocular part of the endoscope, but do not disclose detection of radiation light such as fluorescence radiated from a body tissue. That is, the techniques disclosed in PTLS 1 and 2 are not intended to increase the amount of radiation light received in the diagnosis apparatus as much as possible, and therefore are not provided with the configuration for such a purpose.

Further, an individual difference, between probes, in the connection with the measurement device may be caused by non-uniformity during manufacturing. Therefore, in a diagnosis apparatus, the amount of radiation light received from the probes may be different between the probes. For this reason, before a diagnosis using the probe, it is necessary to adjust the connection between the probe and the measurement device to obtain a maximum amount of light in the diagnosis apparatus. However, the task in association with such adjustment is complicated and troublesome, and therefore forcing the user in the medical field to perform such adjustment has to be avoided as much as possible.

An object of the present invention is to provide an optical measurement device and a probe system which can achieve a highly efficient light connection, that is, which can increase the reception amount of radiation light emitted from a measurement target part of a lumen in a light measurement device, without giving a burden to a user.

### Solution to Problem

An optical measurement device according to an embodiment of the present invention which is connectable to a probe configured to emit measurement light to a measurement target and receive radiation light radiated from the measurement target, the optical measurement device including: a light source of the measurement light; a spectroscope; a first adjustment optical device configured to collect the radiation light received by the probe and emit the radiation light toward the spectroscope configured to divide the radiation light; a detection section configured to detect a light intensity distribution of the radiation light; a movement part configured to move the first adjustment optical device in a light axis direction of the radiation light and on a plane perpendicular to the light axis direction of the radiation light; and a control section configured to control the movement part, wherein the first adjustment optical device is moved in the light axis direction of the radiation light and on the plane perpendicular to the light axis direction of the radiation light on a basis of a detection result of the detection section such that a reception amount of the radiation light increases.

### Advantageous Effects of Invention

According to the present invention, it is possible to increase the reception amount of radiation light emitted from a measurement target part of a lumen in a light measurement device, without giving a burden to a user.

### BRIEF DESCRIPTION OF DRAWINGS

FIG. 1 illustrates a configuration of an endoscope system in an embodiment;

FIG. 2 is a perspective view of an end portion of an endoscope main body of the embodiment;

FIG. 3A illustrates a configuration of a connecting part that connects a probe with a measurement device in the embodiment;

FIG. 3B illustrates a configuration of the connecting part that connects the probe with the measurement device in the embodiment;

FIG. 4 illustrates a state where an optical adjustment of the embodiment is performed;

FIG. 5 illustrates a configuration of a light reception unit of the embodiment;

FIG. 6A illustrates configurations of a measurement light source unit and an illumination light source unit of the embodiment;

FIG. 6B illustrates configurations of the measurement light source unit and the illumination light source unit of the embodiment;

FIG. 7 is a flowchart illustrating an optical adjustment operation of the embodiment;

FIG. 8 is an explanatory view of an astigmatic method of the embodiment;

FIG. 9 is an explanatory view of a movement of the measurement light source unit and a condenser lens of the illumination light source unit of the embodiment;

FIG. 10 illustrates a modification of the configuration of the light reception unit of the embodiment;

FIG. 11 illustrates a modification of the configuration of the light reception unit of the embodiment; and

FIG. 12 illustrates a modification of the configurations of the measurement light source unit and the illumination light source unit of the embodiment.

### DESCRIPTION OF EMBODIMENTS

In the following, the embodiment is described in detail with reference to the drawings.

### Configuration of Endoscope System 1

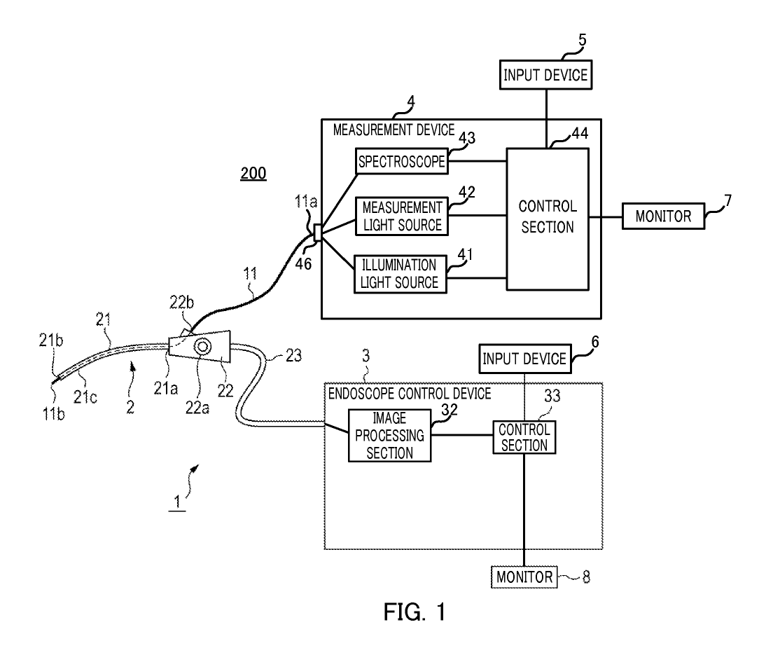

Endoscope system 1 illustrated in FIG. 1 includes: endoscope main body 2 configured to be inserted into a lumen; endoscope control device 3; and probe system 200 for use in examining for a lesion such as cancer and its progression by emitting measurement light to a measurement target part (for example, a lesion) of a lumen and by obtaining radiation light radiated from the measurement target part. Probe system 200 includes probe 11 and measurement device 4 which is connectable to probe 11. As described later, measurement device 4 incorporates an adjustment mechanism for performing optical adjustment. Endoscope main body 2 includes flexible long introduction part 21 which is formed to be capable of being introduced into a lumen, operation part 22 which is provided at a proximal end part 21a of introduction part 21, and cable 23 which communicably connects introduction part 21 with endoscope control device 3 through operation part 22.

Introduction part 21 has, over substantially the entire length thereof, such a flexibility that it can be readily bent to follow the curvature of the lumen when it is advanced in the lumen. In addition, introduction part 21 has a mechanism (not illustrated) which can bend a part (operable part 21c) of distal end part 21b in a certain range at any angle in accordance with operation from nob 22a of operation part 22.

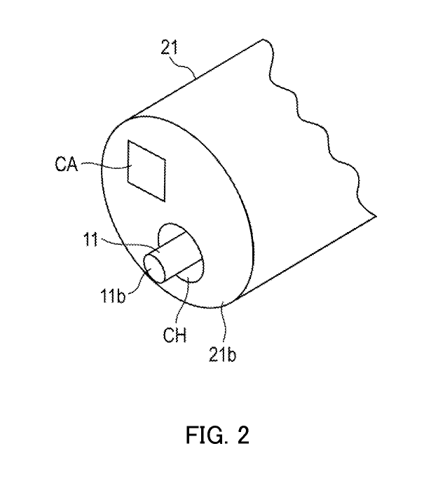

As illustrated in FIG. 2, end part 21b of endoscope main body 2 includes camera CA, forceps channel CH, an air-and-water supply nozzle (not illustrated), and the like.

Camera CA is an electron camera having a solid imaging device. Camera CA images a region illuminated with illumination light, and transmits a resulting image signal to endoscope control device 3.

Forceps channel CH is an inner cavity having a diameter of 2.6 [mm] which is formed in operation part 22 in such a manner as to communicate with introduction part 21 formed in inlet 22b. In forceps channel CH, various devices for observation, diagnosis, and operation of a lesion and the like can be inserted. In the embodiment, as illustrated in FIG. 1, it is possible to insert probe 11 which can examine for a lesion such as cancer and its progression by optical measurement in which light is emitted to a measurement object part in a lumen and light radiated from the measurement target part is acquired. During the optical measurement, probe 11 protrudes from forceps channel CH by about 30 [mm] at maximum.

Probe 11 is intended for single-use, and is a long flexible tubular member extending from probe proximal end portion 11a to probe end portion 11b, as illustrated in FIG. 1. Probe 11 is connected with measurement device 4 through probe connector 46 provided at probe proximal end portion 11a. Probe 11 and measurement device 4 make up probe system 200.

Probe 11 includes therein a measurement optical fiber which guides measurement light, a radiation light optical fiber which receives radiation light, and a illumination fiber which guides illumination light.

The illumination fiber of probe 11 guides illumination light (visible light) emitted from illumination light source 41 of measurement device 4 to probe end portion 11b, and emits the illumination light from probe end portion 11b.

The measurement optical fiber of probe 11 guides measurement light emitted from measurement light source 42 of measurement device 4 to probe end portion 11b, and emits the illumination light from probe end portion 11b.

The light reception fiber of probe 11 receives radiation light radiated from the measurement target part in response to the emission of measurement light, and guides the light to measurement device 4.

### Configuration of Measurement Device 4

Next, a configuration of measurement device 4 will be described. Measurement device 4 includes illumination light source 41 such as an LED which generates illumination light for observation, measurement light source 42 which generates measurement light for measurement, spectroscope 43, and control section 44. Control section 44 controls the operation of each block of measurement device 4. Measurement device 4 is connected with input device 5 and monitor 7.

From input device 5, a user's instruction for measurement device 4 is input. In the embodiment, input device 5 is composed of, for example, a keyboard, mouse, switch or the like. Monitor 7 receives image data output from measurement device 4 to display various kinds of images.

When an instruction for execution of a process for illuminating an observation target part in a lumen is input from input device 5, illumination light source 41 emits illumination light for observation and supplies the light to the illumination fiber of probe 11. When probe 11 has been introduced in a lumen by being inserted into forceps channel CH, probe 11 guides the illumination light emitted from illumination light source 41, and emits the light to the observation target part.

When an instruction for execution of a process for inspecting a measurement target part (biological tissue) in a lumen is input from input device 5, measurement light source 42 emits excitation light such as xenon light and supplies the light to the measurement optical fiber of probe 11. When probe 11 has been introduced in a lumen by being inserted into forceps channel CH, probe 11 guides the light emitted from measurement light source 42, and emits the light as measurement light for the measurement target part. In addition, probe 11 receives light from the measurement target part as biological information of the measurement target part, and guides the light to spectroscope 43 of measurement device 4.

In the embodiment, fluorescence spectroscopy or Raman spectroscopy is employed as the method for measuring a measurement target part. In fluorescence spectroscopy or Raman spectroscopy, laser light having a predetermined wavelength is emitted to a measurement target part as excitation light, and fluorescence or Raman scattering light radiated from the measurement target part in response to the emission of the excitation light is received as radiation light, thereby obtaining a spectral spectrum required for the diagnosis.

From radiation light from a measurement target part guided through the light reception fiber of probe 11, spectroscope 43 measures the intensities of some of wavelengths (hereinafter referred to as “spectrometry measurement”), and outputs the measurement results as a spectroscopic signal.

Control section 44 analyses the spectroscopic signal output from spectroscope 43 to examine for a lesion and its kind in the measurement target part of a lumen. Then, control section 44 outputs diagnosis result image data representing the diagnosis results to monitor 7, and controls image monitor 7 to display the diagnosis result. By visually recognizing the diagnosis result image displayed on monitor 7, a user can evaluate the expansion of the lesion and the degree of the disease.

### Configuration of Endoscope Control Device 3

Next, a configuration of endoscope control device 3 will be described. Endoscope control device 3 is an apparatus for controlling the imaging of endoscope main body 2 in accordance with an operation of a user. Endoscope control device 3 includes image processing section 32 and control section 33. Endoscope control device 3 is connected with input device 6 and monitor 8.

Input device 6 receives a user's instruction for endoscope control device 3. In the embodiment, input device 6 is composed of, for example, a keyboard, mouse, switch or the like. Monitor 8 receives image data output from endoscope control device 3 and displays various kinds of images.

Image processing section 32 receives an imaging signal from endoscope main body 2, and performs a predetermined signal process on the imaging signal, and then, outputs the processed signal to monitor 8 as an endoscope video signal. In this manner, an endoscope image based on the endoscope video signal is displayed on a screen of monitor 8. That is, an image of an observation target part in a lumen is captured, and then the image is displayed on monitor 8. Control section 33 controls the operation of image processing section 32.

### Configuration of Connecting Part which Connects Probe 11 with Measurement Device 4

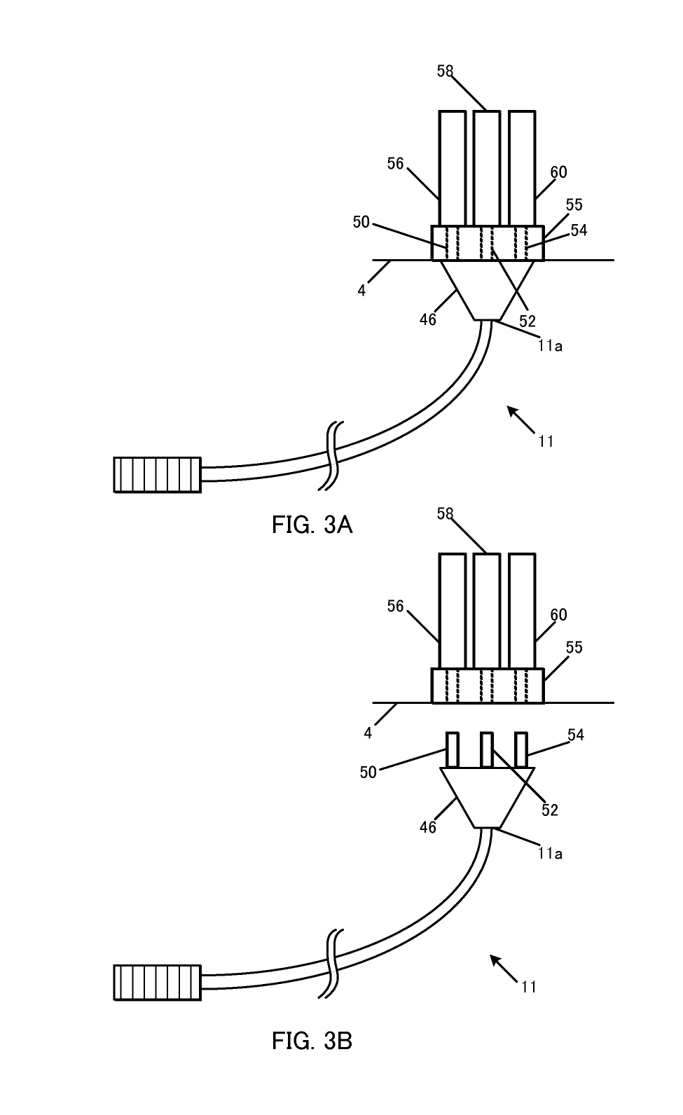

Next, a configuration of a connecting part which connects probe 11 with measurement device 4 will be described. As illustrated in FIGS. 3A and 3B, probe 11 is connected with connector 55 of measurement device 4 through probe connector 46 provided at probe proximal end portion 11a. FIG. 3A illustrates a state where probe 11 is connected with connector 55 of measurement device 4 through probe connector 46. FIG. 3B illustrates a state where probe 11 is separated from connector 55 of measurement device 4. At an end portion of probe connector 46, connector pins 50, 52, and 54 are disposed as connecting terminals for the connection with measurement device 4, and connector pins 50, 52, and 54 serve as a male connector. Connector 55 of measurement device 4 is a female connector configured to receive the above-mentioned connector pins 50, 52 and 54. Next to connector 55 of measurement device 4, measurement light source unit 56, illumination light source unit 58 and light reception unit 60 are disposed in measurement device 4 so as to respectively face connector pins 50, 52 and 54 when probe connector 46 is connected to connector 55.

Connector pin 50 is connected with an end portion of the measurement optical fiber provided in probe 11. Connector pin 50 includes therein a glass fiber. Connector pin 50 guides measurement light emitted from measurement light source 42 of measurement device 4 to the measurement optical fiber provided in probe 11 through measurement light source unit 56.

Connector pin 52 is connected with an end portion of the illumination fiber provided in probe 11. Connector pin 52 includes therein a plastic fiber or glass fiber. Connector pin 52 guides illumination light emitted from illumination light source 41 of measurement device 4 to the illumination optical fiber provided in probe 11 through illumination light source unit 58.

Connector pin 54 is connected with an end portion of the light reception fiber provided in probe 11. Connector pin 54 includes therein a glass fiber. Connector pin 54 guides radiation light received by the light reception fiber provided in probe 11 to light reception unit 60.

Measurement light source unit 56 includes measurement light source 42, and a measurement light optical system for guiding measurement light emitted by measurement light source 42 to connector pin 50. The measurement light optical system is provided with a configuration for performing a measurement light adjustment operation for maximizing the amount of measurement light passing through measurement light source unit 56, in the state where probe 11 and measurement device 4 are connected with each other.

Illumination light source unit 58 includes illumination light source 41, and an illumination light optical system for guiding illumination light emitted by illumination light source 41 to connector pin 52. The illumination light optical system is provided with a configuration for performing an illumination light adjustment operation for maximizing the amount of illumination light passing through illumination light source unit 58, in the state where probe 11 and measurement device 4 are connected with each other.

Light reception unit 60 includes a light reception optical system for guiding radiation light guided from connector pin 54 to spectroscope 43 of measurement device 4. The light reception optical system is provided with a configuration for performing a light reception adjustment operation for maximizing the amount of radiation light passing through light reception unit 60, in the state where probe 11 and measurement device 4 are connected with each other.

It is to be noted that, in FIG. 3, measurement light source unit 56, illumination light source unit 58, light reception unit 60 are simplified.

### Configuration of Reflection Member Tool 70

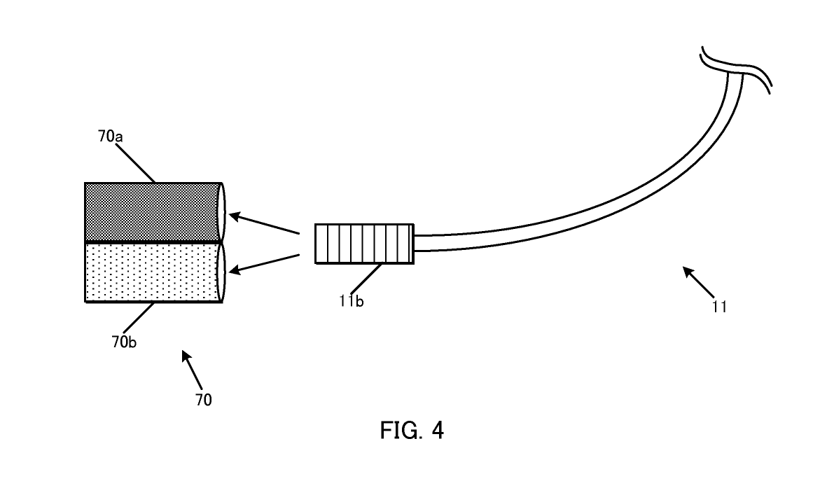

In the embodiment, probe end portion 11b is covered with reflection member tool 70 having a cap shape prior to the measurement light adjustment operation, the illumination light adjustment operation and the light reception adjustment operation, as illustrated in FIG. 4. Then, light (measurement light or illumination light) is emitted from probe 11 to the inside of reflection member tool 70, and the reflection light is received by probe 11. It is to be noted that reflection member tool 70 may be a separate member, or may be provided in measurement device 4.

Reflection member tool 70 includes first reflection member tool 70a and second reflection member tool 70b. A black sheet is provided in first reflection member tool 70a so that the light emitted from probe 11 is not reflected. When light is emitted in the state where probe end portion 11b is inserted in first reflection member tool 70a, light which is unnecessary for the measurement light adjustment operation, the illumination light adjustment operation and the light reception adjustment operation (hereinafter referred to as “unnecessary light”) is detected by detecting the light received by probe 11. Examples of the unnecessary light include external light, reflection light from a lens provided in probe 11, and the like. The unnecessary light detected here is used in the measurement light adjustment operation, the illumination light adjustment operation and the light reception adjustment operation.

In second reflection member tool 70b, a sheet (for example, Munsell sheet) whose reflectance is known is provided. That is, light emitted from probe 11 is reflected by the sheet, and received by probe 11. By using the light thus received, the measurement light adjustment operation, the illumination light adjustment operation and the light reception adjustment operation are performed. It is to be noted that the light received by probe 11 contains unnecessary light which may have a negative influence on the adjustment operations, and therefore the adjustment operations are performed after the unnecessary light is removed from the received light. The following descriptions will be made on the premise that the adjustment operations are performed after detecting and removing unnecessary light.

In the measurement light adjustment operation, measurement light is emitted to the inside of second reflection member tool 70b through measurement light source unit 56, and the reflection light (measurement light) is received by probe 11. The reflection light received by probe 11 is detected by light reception unit 60, and, on the basis of the detection results, optical adjustment is performed for the measurement light optical system of measurement light source unit 56.

In the illumination light adjustment operation, illumination light is emitted to the inside of second reflection member tool 70b through illumination light source unit 58, and the reflection light (illumination light) is received by probe 11. The reflection light received by probe 11 is detected by light reception unit 60, and, on the basis of the detection results, optical adjustment is performed for the illumination light optical system of illumination light source unit 58.

In the light reception adjustment operation, measurement light is emitted to the inside of second reflection member tool 70b through measurement light source unit 56, and the reflection light (illumination light) is received by probe 11. The reflection light received by probe 11 is detected by light reception unit 60, and, on the basis of the detection results, optical adjustment is performed for the light reception optical system of light reception unit 60.

### Configuration of Light Reception Unit 60

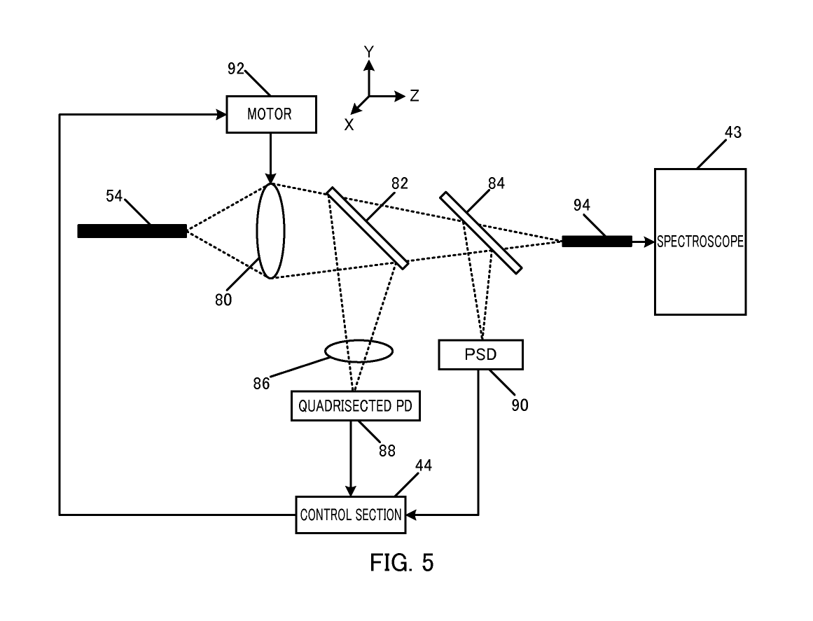

Next, a configuration of light reception unit 60 will be described. As illustrated in FIG. 5, light reception unit 60 includes condenser lens 80 which functions as a first adjustment optical device, half mirror 82 which functions as a first branching optical element, half minor 84 which functions as a second branching optical element, cylindrical lens 86, quadrisected photodetector 88 (hereinafter referred to as “quadrisected PD”) which functions as a first detection sensor, position sensitive detector 90 (hereinafter referred to as “PSD”) which functions as a second detection sensor, and motor 92. Motor 92 functions as a first movement part, a second movement part and a rotation part.

Condenser lens 80 is a biconvex lens. Condenser lens 80 collects radiation light guided by connector pin 54 and emits the light toward half mirror 82.

Part of radiation light emitted from condenser lens 80 is transmitted through half mirror 82, and half mirror 82 reflects the other part of the radiation light toward cylindrical lens 86 such that the other part of the radiation light branches from the light path of the radiation light.

Part of radiation light which has been transmitted through half mirror 82 is transmitted through half mirror 84, and half mirror 84 reflects the other part of the radiation light toward PSD 90 such that the other part of the radiation light is divided from the light path of the radiation light.

Together with quadrisected PD 88, cylindrical lens 86 makes up an astigmatism optical system. Cylindrical lens 86 gives astigmatism to radiation light reflected by half minor 82 and causes the light to impinge on quadrisected PD 88.

Quadrisected PD 88 receives radiation light from cylindrical lens 86, detects the light intensity distribution of the received radiation light on a plane perpendicular to the light axis direction of the radiation light, and outputs a detection signal to control section 44.

PSD 90 receives radiation light reflected by half mirror 84, detects the light intensity distribution of the radiation light in one direction on a plane perpendicular to the light axis direction of the received radiation light, and outputs a detection signal to control section 44. From the detection result of PSD 90, the gravity center of the radiation light can be computed.

Motor 92 is composed of five stepping motors, and, under the control of control section 44, moves condenser lens 80 in the light axis direction of radiation light (the Z-axis direction in the drawing) and on a plane perpendicular to the light axis direction (the plane defined by the X-axis direction and the Y-axis direction in the drawing). In addition, under the control of control section 44, motor 92 rotates condenser lens 80 around the direction perpendicular to the light axis direction of radiation light (the X-axis direction and the Y-axis direction in the drawing).

Radiation light having been transmitted through half minor 84 is guided to spectroscope 43 by spectroscope fiber 94.

It is to be noted that a dichroic mirror may also be used instead of half mirrors 82 and 84. When a dichroic minor is used, the efficiency of the spectrometry measurement in spectroscope 43 can be enhanced by selecting a minor configured to reflect light having a wavelength region which is not subjected to the spectrometry measurement of spectroscope 43.

### Configuration of Measurement Light Source Unit 56

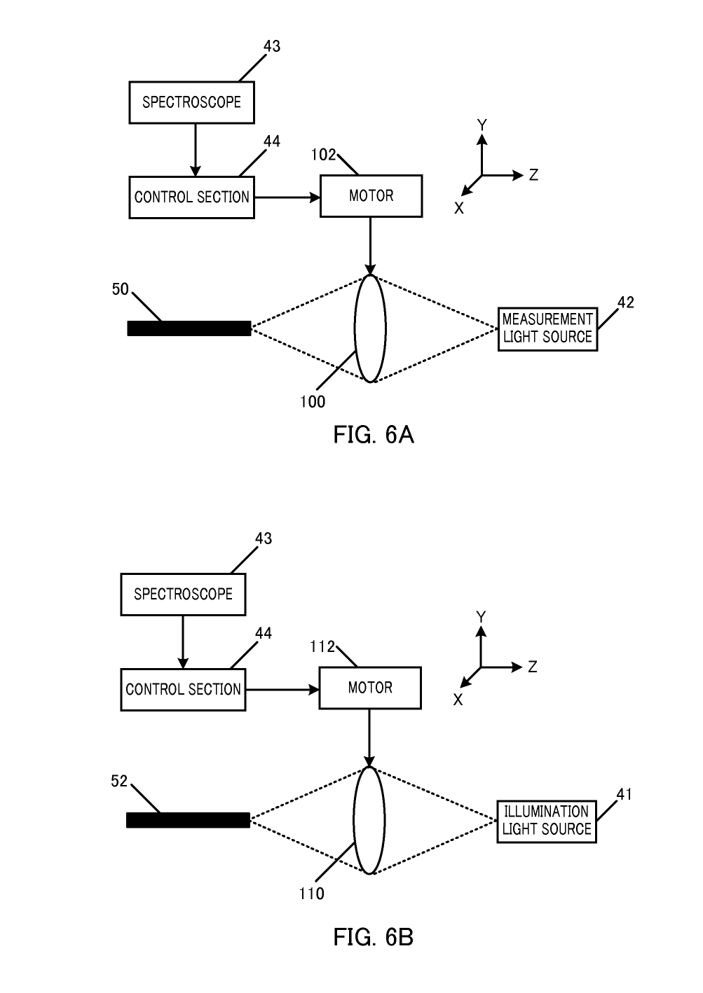

Next, a configuration of measurement light source unit 56 will be described. As illustrated in FIG. 6A, measurement light source unit 56 includes condenser lens 100 which functions as a second adjustment optical device, and motor 102 which functions as a third movement part and a fourth movement part.

Condenser lens 100 is a biconvex lens. Condenser lens 100 collects measurement light emitted by measurement light source 42 and emits the measurement light toward connector pin 50.

Motor 102 is composed of five stepping motors, and, under the control of control section 44, moves condenser lens 100 in the light axis direction of measurement light (the Z-axis direction in the drawing) and on a plane perpendicular to the light axis direction (the plane defined by the X-axis direction and the Y-axis direction in the drawing). In addition, under the control of control section 44, motor 102 rotates condenser lens 100 around the direction perpendicular to the light axis direction of measurement light (the X-axis direction and the Y-axis direction in the drawing).

### Configuration of Illumination Light Source Unit 58

Next, a configuration of illumination light source unit 58 will be described. As illustrated in FIG. 6B, illumination light source unit 58 includes condenser lens 110 which functions as a third adjustment optical device, and motor 112 which functions as a fifth movement part and a sixth movement part.

Condenser lens 110 is a biconvex lens. Condenser lens 110 collets illumination light emitted by illumination light source 41 and emits the illumination light toward connector pin 52.

Motor 112 is composed of five stepping motors, and, under the control of control section 44, moves condenser lens 110 in the light axis direction of illumination light (the Z-axis direction in the drawing) and on a plane perpendicular to the light axis direction (the plane defined by the X-axis direction and the Y-axis direction in the drawing). In addition, under the control of control section 44, motor 112 rotates condenser lens 110 around the direction perpendicular to the light axis direction of illumination light (the X-axis direction and the Y-axis direction in the drawing).

It is to be noted that motors 92, 102 and 112 of light reception unit 60, measurement light source unit 56, illumination light source unit 58 may be composed of a DC motor, a servomotor, a voice coil motor (VCM), a piezoelectric ultrasonic linear actuator (SIDM) or the like instead of the stepping motor. In addition, the driving amount of condenser lenses 80, 100 and 110 through motors 92, 102 and 112 may be controlled by using a position detection sensor such as a linear sensor and an encoder. In particular, when the driving amount of condenser lenses 80, 100 and 110 is a small amount not greater than [μm], a position detection sensor having an optical system such as a length measuring machine is preferably used for the control.

### Optical Adjustment Operation in Probe System 200

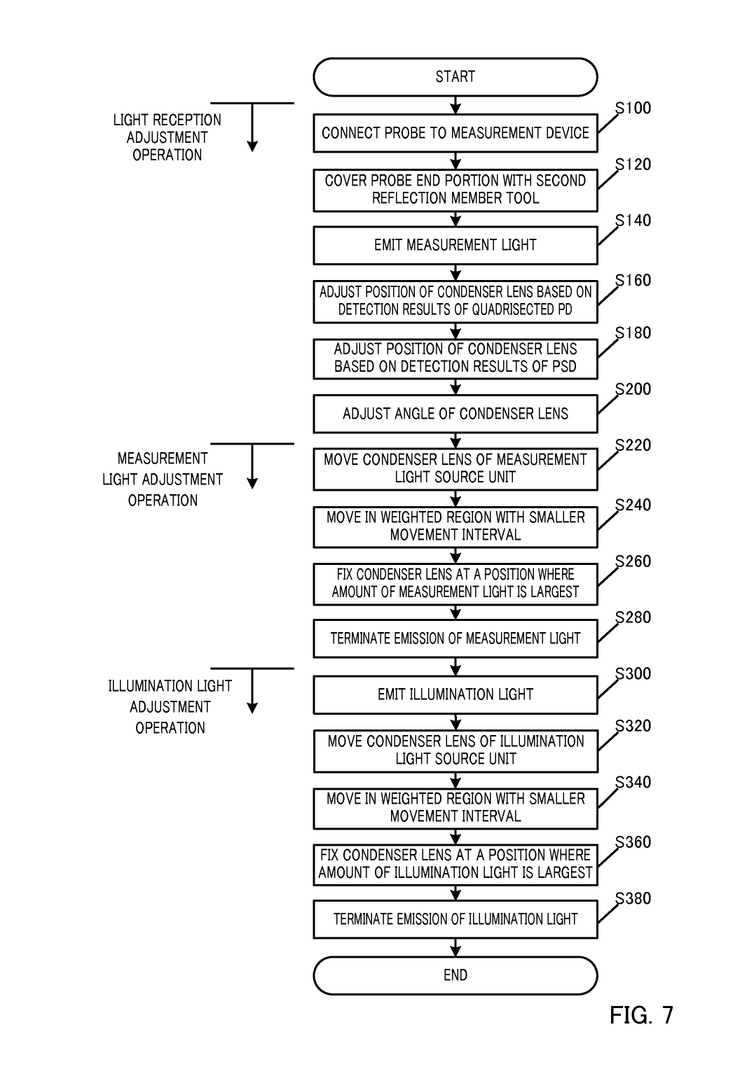

Next, referring to the flowchart of FIG. 7, an optical adjustment operation in probe system 200 will be described. In the embodiment, the optical adjustment operation is an operation in which the light reception adjustment operation, the measurement light adjustment operation and the illumination light adjustment operation are continuously performed in the mentioned order.

First, a user connects probe 11 to measurement device 4 (step S100). Next, the user covers probe end portion 11b with second reflection member tool 70b (step S120).

Next, when an instruction for execution of the optical adjustment operation is input from input device 5 of measurement device 4, control section 44 controls measurement light source 42 to emit measurement light (step S140). Probe 11 guides measurement light emitted from measurement light source 42 and emits the measurement light to the inside of second reflection member tool 70b. In addition, probe 11 receives radiation light from the sheet provided in second reflection member tool 70b, and guides the light to light reception unit 60.

Next, control section 44 controls motor 92 on the basis of the detection results of radiation light of quadrisected PD 88 of light reception unit 60, and adjusts the position of condenser lens 80 in the Z direction in the drawing (see FIG. 5) (step S160).

In the embodiment, an astigmatic method is used for the position adjustment of condenser lens 80 in the Z direction in the drawing. First, radiation light emitted from condenser lens 80 is divided by half mirror 82, and part of the radiation light is caused to impinge on quadrisected PD 88 through cylindrical lens 86. Since cylindrical lens 86 is effective only for light in a certain one direction (polarization direction), the focus position on the light reception surface of quadrisected PD 88 in the vertical axis direction and the lateral axis direction is shifted. Therefore, the shape of the radiation light having passed through cylindrical lens 86 is a vertically long ellipse, a circle, or a laterally long ellipse, and varies depending on the position of condenser lens 80 on the light axis of the radiation light.

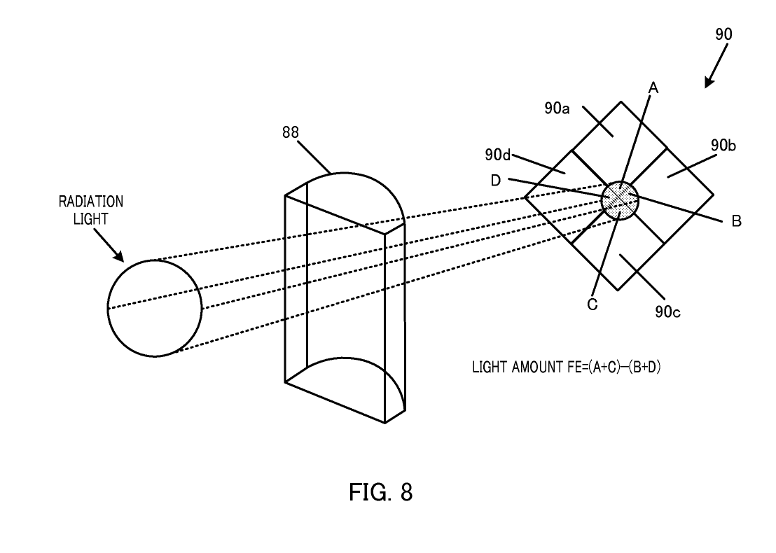

As illustrated in FIG. 8, when four photodiodes divided by quadrisected PD 88 are represented by 90a, 90b, 90c and 90d, and signals output from 90a, 90b, 90c and 90d are represented by A, B, C and D, respectively, light amount FE used for the position adjustment of condenser lens 80 in the Z direction in the drawing is expressed by the following Expression (1).

Light amount FE =(A+C)−(B+D) (1)

As shown by Expression (1), light amount FE is obtained by calculating the sums of the output signals from a pair of diagonally disposed photodiodes, and by calculating the difference thereof.

When the shape of radiation light having passed through cylindrical lens 86 is a circle, light amount FE is 0. By moving condenser lens 80 to a position where light amount FE is 0, the spot position of condenser lens 80 can be set to a position that matches the position of spectroscope fiber 94.

Next, on the basis of the detection result of the radiation light of PSD 90 of light reception unit 60, control section 44 controls motor 92 to move condenser lens 80 on a plane perpendicular to the light axis direction of radiation light (the plane defined by the X-axis direction and the Y-axis direction in the drawing) (step S180).

To be more specific, radiation light having been transmitted through half mirror 82 is divided by half mirror 84, and part of the radiation light is caused to impinge on PSD 90. In the embodiment, the correlation between the gravity center of radiation light computed from the detection result of PSD 90, and the reception position of the radiation light at spectroscope 43 is determined in advance at the time of manufacturing measurement device 4. Thus, by moving condenser lens 80 on a plane perpendicular to the light axis direction of radiation light such that the gravity center determined in advance matches the gravity center of the radiation light computed from the detection result of PSD 90, it is possible to set the light reception position of radiation light at spectroscope 43 to a position that matches the spot position of condenser lens 80.

Next, control section 44 controls motor 92 to rotate condenser lens 80 around the direction perpendicular to the light axis direction of radiation light (the X-axis direction and the Y-axis direction in the drawing) (step S200).

When condenser lens 80 is moved on a plane perpendicular to the light axis direction of radiation light, condenser lens 80 moves to the outside of the light axis relative to measurement light source 42 and spectroscope 43. Thus, image height is generated, and the shape of the light spot of condenser lens 80 is changed to a shape which is not a precise circle. In addition, an aberration such as comatic aberration and saddle-type aberration which degrades the shape of the light spot is generated. Such an aberration is desirably canceled as much as possible, and can be canceled by rotating (tilting) condenser lens 80 around the direction perpendicular to the light axis direction of radiation light. Through the processes of steps S160 to S200, the amount of radiation light which impinges on spectroscope 43 after passing through light reception unit 60 can be maximized.

Since the aberration generated when condenser lens 80 moves to the outside of the light axis can be simulated at the time of design, the amount of aberration generated in accordance with the movement, and the rotational angle of condenser lens 80 (lens tilting amount) required for cancelling the aberration are calculated and stored in advance in the embodiment. After condenser lens 80 is moved on a plane perpendicular to the light axis direction of radiation light, the lens tilting amount for cancelling the aberration generated in accordance with the movement is read out, and condenser lens 80 is tilted by the lens tilting amount. The movement on a plane perpendicular to the light axis direction of radiation light and the rotation around the direction perpendicular to the light axis direction of the radiation light may be simultaneously performed such that the path of condenser lens 80 forms an arc-like shape.

While condenser lens 80 is adjusted through the procedures of steps S160 to S200, the following procedures of (i) to (iii) are used to move condenser lens 100 of measurement light source unit 56 until radiation light is detected by quadrisected PD 88 in the case where radiation light guided by connector pin 54 cannot be detected by quadrisected PD 88 at step S160. Naturally, when no radiation light impinges on light reception unit 60 of probe 11, condenser lens 80 cannot be adjusted by a servo control. In this case, it is presumed that some problems have occurred in the measurement light optical system of measurement light source unit 56. Therefore, before adjusting condenser lens 80 in light reception unit 60, a rough measurement light adjustment operation is required to be performed in measurement light source unit 56 such that at least the light reception adjustment operation can be performed.
(i) Condenser lens 100 is moved on a plane perpendicular to the light axis direction. At this time, the angle of condenser lens 100 is fixed.
(ii) When radiation light cannot be detected by quadrisected PD 88 after condenser lens 100 is moved, condenser lens 100 is rotated in a movable range at each point of the movement of condenser lens 100.
(iii) When radiation light can be detected by quadrisected PD 88 after the movement and rotation of condenser lens 100, the process is returned to step S160. When radiation light cannot be detected by quadrisected PD 88 after the movement and rotation of condenser lens 100, control section 44 determines that an error such as defect of probe 11 which does not occur in a normal configuration has occurred, and returns an error message. Then, the optical adjustment operation in probe system 200 is terminated.

Returning back to the flowchart of FIG. 7, at steps S220 to S260, the measurement light adjustment operation in measurement light source unit 56 is performed. That is, measurement light source unit 56 of high importance is first adjusted, and then illumination light source unit 58 is adjusted. To adjust the position of condenser lens 100 in measurement light source unit 56, measurement light source 42 emits measurement light even after the adjustment of the position of condenser lens 80 in light reception unit 60 is completed. The measurement of the amount of the measurement light which is performed to adjust the position of condenser lens 100 in measurement light source unit 56 is achieved by using spectroscope 43 of measurement measurement device 4. In the measurement of the amount of the measurement light using spectroscope 43, a constant light exposure time of spectroscope 43 is set, and the sum of the light intensity of each wavelength of the measured light (radiation light) is obtained as the light amount.

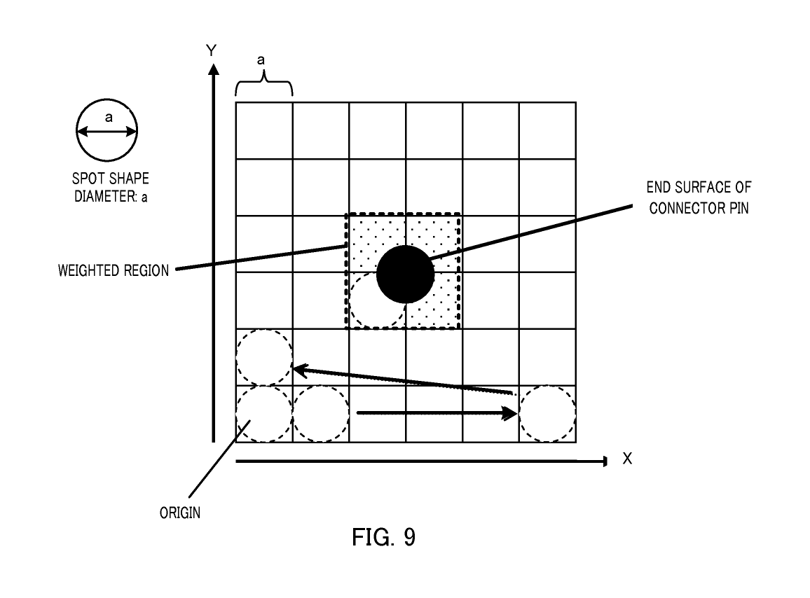

At step S220, control section 44 controls motor 102 to move the position of condenser lens 100 in measurement light source unit 56, in the state where measurement of the amount of measurement light is performed in spectroscope 43. To be more specific, first, condenser lens 100 is moved to the origin on a plane perpendicular to the light axis direction of measurement light (the plane defined by the X-axis direction and the Y-axis direction in the drawing) as illustrated in FIG. 9. Thereafter, condenser lens 100 is moved to a predetermined position in the Z-axis direction. In this case, the size (diameter: a) of the spot emitted from the position of condenser lens 100 is obtained in advance, and condenser lens 100 is moved on the spot size basis along the arrow direction in the drawing, that is, in a mesh form (grid form).

The size of the spot is determined in the following manner. That is, when probe 11 emits measurement light and receives radiation light, condenser lens 100 is moved in the light axis direction of the measurement light, and the diameter of a region where the light intensity of the radiation light incident on spectroscope 43 is a predetermined value is defined as the size of the spot (hereinafter referred to as “first spot diameter”). The first spot diameter is stored in the form of data, and is read out at the time of the measurement light adjustment operation. The predetermined value is 1/e², or more preferably, ½ of the peak of the light intensity of measurement light computed on the basis of the reflectance of the sheet provided in second reflection member tool 70b.

Condenser lens 100 is moved on the first spot diameter basis on a plane perpendicular to the light axis of the measurement light, and the position of condenser lens 100 where the light intensity of the radiation light incident on spectroscope 43 is greatest is determined. With the determined position at the center, the area where the end surface of connector pin 50 is possibly located (hereinafter referred to as “weighted region”) is set from the size of the end surface of connector pin 50, and condenser lens 100 is moved on a diameter smaller than the first spot diameter basis (step S240) in the weighted region, including the Z-axis direction.

Next, the position of condenser lens 100 where the light intensity of the radiation light incident on spectroscope 43 is greatest is determined, and condenser lens 100 is fixed at the determined position (step S260). At this time, at the time when condenser lens 100 is moved on a plane perpendicular to the light axis direction of the measurement light, condenser lens 100 is rotated around the direction perpendicular to the light axis direction of the measurement light so as to cancel off-axial aberrations such as comatic aberration and astigmatism caused by the movement of condenser lens 100.

By performing the measurement light adjustment operation by the method of steps S220 to S260, the position where measurement light impinges on connector pin 50 (fiber) can be detected with a small number of measurement points, and highly accurate adjustment can be performed in a short time.

It is to be noted that, when measurement light cannot be detected by spectroscope 43 during the movement of condenser lens 100, it is possible to display an error message on monitor 7 to facilitate the user to reconnect probe 11 with measurement device 4. Further, when measurement light cannot be detected by spectroscope 43 even after reconnection, it is possible to display on monitor 7 an image that requests the user to replace probe 11. When the error is displayed even after replacement, it is possible to display a massage that facilitates the user to contact service man, since there is a possibility of malfunction of the apparatus main body or the like.

Next, control section 44 controls measurement light source 42 to terminate emission of measurement light (step S280). Control section 44 controls illumination light source 41 to emit illumination light (step S300).

Control section 44 controls motor 112 to move the position of condenser lens 110 in illumination light source unit 58 in the state where measurement of the amount of illumination light is performed in spectroscope 43 (step S320). To be more specific, first, condenser lens 110 is moved to the origin on a plane perpendicular to the light axis direction of the illumination light (the plane defined by the X-axis direction and the Y-axis direction in the drawing) as illustrated in FIG. 9. Thereafter, condenser lens 110 is moved to a predetermined position in the Z-axis direction. In this case, the size (diameter: a) of the spot emitted from the position of condenser lens 110 is obtained in advance, and condenser lens 100 is moved on the spot size basis along the arrow direction in the drawing.

The size of the spot is determined in the following manner. That is, when probe 11 emits illumination light and receives illumination light, condenser lens 110 is moved in the light axis direction of the illumination light, and the diameter of a region where the light intensity of the illumination light incident on spectroscope 43 is a predetermined value is defined as the size of the spot (hereinafter referred to as “second spot diameter”). The second spot diameter is stored in the form of data, and is read out at the time of the illumination light adjustment operation. The predetermined value is 1/e², or more preferably, ½ of the peak of the light intensity of illumination light computed on the basis of the reflectance of the sheet provided in second reflection member tool 70b.

Condenser lens 110 is moved on the second spot diameter basis on a plane perpendicular to the light axis of the illumination light, and the position of condenser lens 110 where the light intensity of the illumination light incident on spectroscope 43 is greatest is determined. With the determined position at the center, the area where the end surface of connector pin 52 is possibly located (hereinafter referred to as “weighted region”) is set from the size of the end surface of connector pin 52, and condenser lens 110 is moved on a diameter smaller than the second spot diameter basis (step S340) in the weighted region, including the Z-axis direction.

Next, the position of condenser lens 110 where the light intensity of the illumination light incident on spectroscope 43 is greatest is determined, and condenser lens 110 is fixed at the determined position (step S260). At this time, at the time when condenser lens 110 is moved on a plane perpendicular to the light axis direction of the illumination light, condenser lens 110 is rotated around the direction perpendicular to the light axis direction of the illumination light so as to cancel off-axial aberrations such as comatic aberration and astigmatism caused by the movement of condenser lens 110.

By performing the illumination light adjustment operation by the method of steps S320 to S360, the position where illumination light impinges on connector pin 52 (fiber) can be detected with a small number of measurement points, and highly accurate adjustment can be performed in a short time.

It is to be noted that, when illumination light cannot be detected by spectroscope 43 during the movement of condenser lens 110, it is possible to display an error message on monitor 7 to facilitate the user to reconnect probe 11 with measurement device 4. Further, when illumination light cannot be detected by spectroscope 43 even after reconnection, it is possible to display on monitor 7 an image that requests the user to replace probe 11. When the error is displayed even after replacement, it is possible to display a massage that facilitates the user to contact service man.

Finally, control section 44 controls illumination light source 41 to terminate the emission of illumination light (step S380). Upon completion of the process of step S380, the optical adjustment operation of in FIG. 7 is completed.

### Effect of Embodiment

As has been described in detail, the optical measurement device of the embodiment includes: a first adjustment optical device (condenser lens 80) configured to collect radiation light received by probe 11 and emit the radiation light toward divide spectroscope 43; a first detection section configured to detect a light intensity distribution of radiation light on a plane perpendicular to the light axis direction of the radiation light; a second detection section configured to detect the light intensity distribution of the radiation light in one direction on the plane; a first movement part (motor 92) configured to move the first adjustment optical device in the light axis direction on the basis of detection results of the first detection section; a second movement part (motor 92) configured to move the first adjustment optical device on the plane on the basis of detection results of the second detection section; and control section 44 configured to control a first movement part and a second movement part. Probe 11 is configured to emit measurement light to a measurement target (Munsell sheet) whose reflectance is known, and receive radiation light radiated from the measurement target. Thus, the position of condenser lens 80 is automatically adjusted on the basis of detection results of the first detection section and the second detection section such that the amount of radiation light incident on spectroscope 43 is maximized. Consequently, without giving a burden to a user, it is possible to increase the reception amount of radiation light emitted from a measurement target part of a lumen in light measurement device 4. In addition, since the light reception adjustment operation, the measurement light adjustment operation and the illumination light adjustment operation are performed in the mentioned order, automatic adjustment can be efficiently performed.

In addition, in the embodiment, probe 11 and measurement device 4 are connected with each other through a plurality of connecting terminals. In general, when a plurality of male structures and female structures are fitted to each other, it is difficult to tightly fit the structures without gap. Typically, considering manufacturing error, such a problem is solved by providing play at some of the fitting parts. In such a configuration, the play results in an individual difference in the connection between the connector of probe 11 and the connector of measurement device 4. In such a case, the configuration of the embodiment, which can automatically perform the reception light adjustment, is further useful.

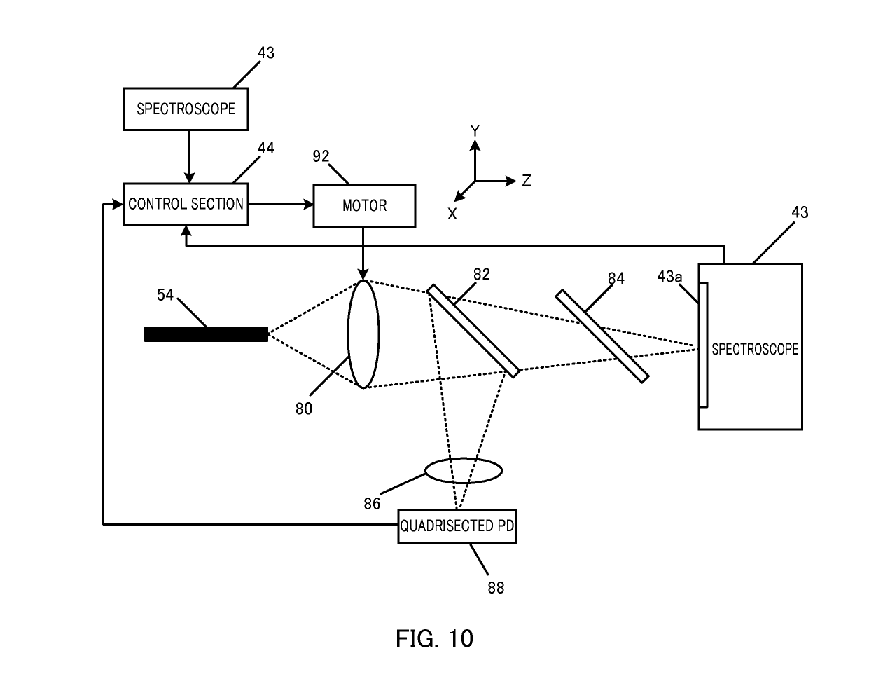

While, in the above-mentioned embodiment, PSD 90 detects the light intensity distribution of radiation light in one direction on a plane perpendicular to the light axis direction of the radiation light, the present invention is not limited to this. For example, as illustrated in FIG. 10, it is possible to use spectroscope 43 having two-dimensional imaging device 43a (for example, CCD (Charge Coupled Device) or CMOS (Complementary Metal Oxide Semiconductor) the like) that receives radiation light and detects the light intensity distribution of the radiation light in one direction on a plane perpendicular to the light axis direction of the radiation light. Spectroscope 43 having two-dimensional imaging device 43a can determine information of the position in imaging device 43a where radiation is incident on imaging device 43a, and the gravity center of the incident radiation light. Therefore, by preliminarily setting the position on two-dimensional imaging device 43a where radiation light is incident, condenser lens 80 can be moved such that the incident position of radiation light is moved to a predetermined position with only the information from two-dimensional imaging device 43a. It is to be noted that a one-dimensional imaging device may be employed instead of two-dimensional imaging device 43a.

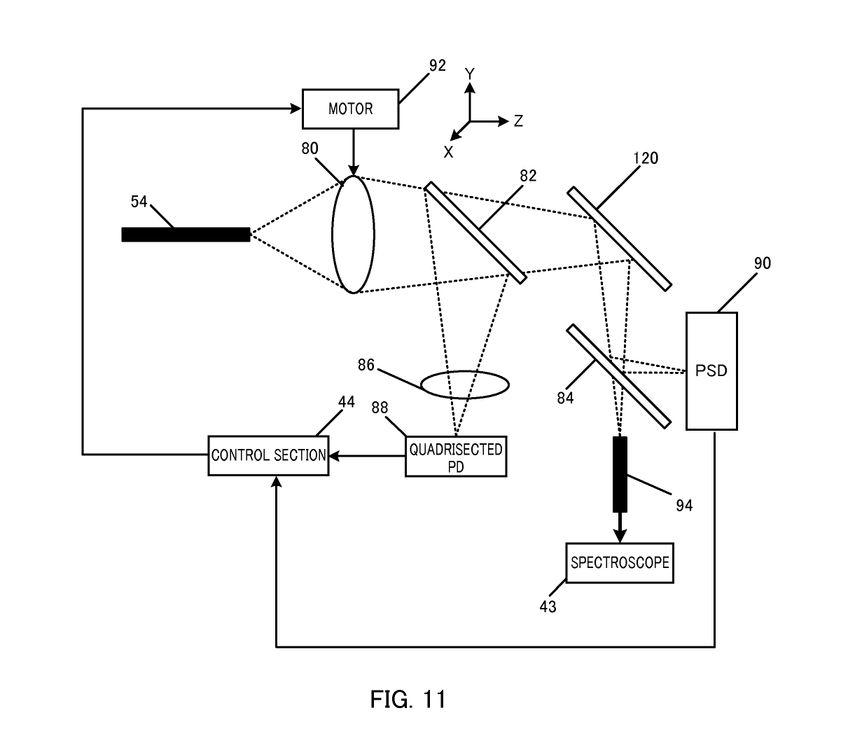

In addition, in the above-mentioned embodiment, it is also possible to provide galvano minor 120 between half mirror 82 and half minor 84 of light reception unit 60 as illustrated in FIG. 11. A MEMES (micro electro mechanical system) mirror may be provided instead of galvano mirror 120. In this case, instead of moving condenser lens 80 on a plane perpendicular to the light axis direction of radiation light (the plane defined by the X-axis direction and the Y-axis direction in the drawing), galvano mirror 120 is rotated around the Y-axis direction in the drawing.

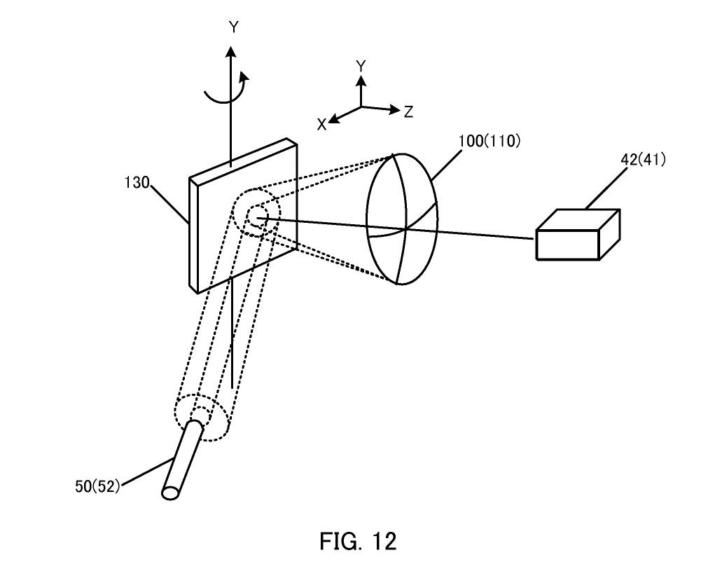

In addition, in the above-mentioned embodiment, it is possible to provide galvano minor 130 between condenser lens 100 (condenser lens 110) and connector pin 50 (connector pin 52) of measurement light source unit 56 (illumination light source unit 58) as illustrated in FIG. 12. A MEMES minor may be provided instead of galvano mirror 130. In this case, instead of moving condenser lenses 100 and 110 on the plane (the plane defined by the X-axis direction and the Y-axis direction in the drawing) perpendicular to the light axis direction of measurement light (illumination light), galvano mirror 130 is rotated around the Y-axis direction in the drawing.

While, in the above-mentioned embodiment, all of the measurement light adjustment operation, the illumination light adjustment operation and the light reception adjustment operation are performed, the present invention is not limited to this. For example, the importance of the adjustment operations decreases in the following order: the light reception adjustment operation, the measurement light adjustment operation, and the illumination light adjustment operation. Thus, it is possible to achieve a configuration that prioritizes the light reception adjustment operation, or more specifically, a configuration in which only the light reception adjustment operation of the highest importance is performed without performing the measurement light adjustment operation and the illumination light adjustment operation.

In addition, in the above-mentioned embodiment, all of connector pins 50, 52 and 54 may have a plastic fiber as long as a minimum required amount of each of measurement light, illumination light and radiation light is ensured.

In addition, in the above-mentioned embodiment, when the spectrometry measurement may possibly influenced by reduction in the amount of radiation light having passed through light reception unit 60 due to half minors 82 and 84, it is possible to move, after firstly performing an optical adjustment such as the light reception adjustment operation, half minors 82 and 84 by an actuator such as a stepping motor to a position which is obtained through a preliminary simulation and which has no influence on the spectrometry measurement. In such a case, since the light path of radiation light is changed when half minors 82 and 84 are moved, it is necessary to correct condenser lens 80 by an amount corresponding to the change.

In addition, in the above-mentioned embodiment, when the luminance distributions of measurement light source 42 and illumination light source 41 are known, the region where the illuminance is highest in the light spot can be determined, and therefore, it is possible to dispose connector pins 50 and 52 in the region to perform the measurement light adjustment operation and the illumination light adjustment operation. With this configuration, it is possible to reduce the amount of movement of condenser lenses 100 and 110 in the measurement light adjustment operation and the illumination light adjustment operation, and to perform the operations in a short time.

The embodiments disclosed herein are merely exemplifications and should not be considered as limitative. While the invention made by the present inventor has been specifically described based on the preferred embodiments, it is not intended to limit the present invention to the above-mentioned preferred embodiments but the present invention may be further modified within the scope and spirit of the invention defined by the appended claims.

This application is entitled to and claims the benefit of Japanese Patent Application No. 2012-209849 filed on Sep. 24, 2012, the disclosure of which including the specification, drawings and abstract is incorporated herein by reference in its entirety.

### REFERENCE SIGNS LIST

1 Endoscope system

2 Endoscope main body

3 Endoscope control device

4 Measurement device

5, 6 Input device

7, 8 Monitor

11 Probe

11a Probe proximal end portion

11b Probe end portion

21 Introduction part

21a Proximal end portion

21b End portion

21c Operable part

22 Operation part

22a Nob

22b Inlet

23 Cable

32 Image processing section

33, 44 Control section

41 Illumination light source

42 Measurement light source

43 Spectroscope

43a Two-dimensional imaging device

46 Probe connector

50, 52, 54 Connector pin

55 Connector

56 Measurement light source unit

58 Illumination light source unit

60 Light reception unit

70 Reflection member tool

70a First reflection member tool

70b Second reflection member tool

80, 100, 110 Condenser lens

82, 84 Half mirror

86 Cylindrical lens

88 Quadrisected PD

90 PSD

90a, 90b, 90c, 90d Photodiode

92, 102, 112 Motor

94 Spectroscope fiber

120, 130 Galvano minor

200 Probe system

CH Forceps channel

CA Camera

1. An optical measurement device which is connectable to a probe configured to emit measurement light to a measurement target and receive radiation light radiated from the measurement target,
the optical measurement device comprising:
a light source of the measurement light;
a spectroscope;
a first adjustment optical device configured to collect the radiation light received by the probe and emit the radiation light toward the spectroscope configured to divide the radiation light;
a detection section configured to detect a light intensity distribution of the radiation light;
a movement part configured to move the first adjustment optical device in a light axis direction of the radiation light and on a plane perpendicular to the light axis direction of the radiation light; and
a control section configured to control the movement part, wherein
the first adjustment optical device is moved in the light axis direction of the radiation light and on the plane perpendicular to the light axis direction of the radiation light on a basis of a detection result of the detection section such that a reception amount of the radiation light increases.

2. The optical measurement device according to claim 1, wherein
the detection section includes
a first detection section configured to detect a light intensity distribution of the radiation light on the plane perpendicular to the light axis direction of the radiation light, and
a second detection section configured to detect a light intensity distribution of the radiation light in one direction on the plane,
the movement part includes
a first movement part configured to move the first adjustment optical device in the light axis direction on a basis of a detection result of the first detection section, and
a second movement part configured to move the first adjustment optical device on the plane on a basis of a detection result of the second detection section, and
control section controls the first movement part and the second movement part.

3. The optical measurement device according to claim 2, wherein
the first detection section includes
a first branching optical element configured to divide a light path of the radiation light,
a cylindrical lens, and
a first detection sensor configured to receive the radiation light through the first branching optical element and the cylindrical lens, and detect the light intensity distribution of the radiation light on the plane.

4. The optical measurement device according to claim 2, wherein
the second detection section includes
a second branching optical element configured to divide a light path of the radiation light, and
a second detection sensor configured to receive the radiation light through the second branching optical element, and detect the light intensity distribution of the radiation light in one direction on the plane.

5. The optical measurement device according to claim 4, wherein the second detection sensor includes an imaging device including pixels which are two-dimensionally disposed, the imaging device being configured to receive the radiation light and detect the light intensity distribution of the radiation light on the plane.

6. The optical measurement device according to claim 4, wherein the second detection sensor includes an imaging device including pixels which are one-dimensionally disposed, the imaging device being configured to receive the radiation light and detect the light intensity distribution of the radiation light in one direction on the plane.

7. The optical measurement device according to claim 1 further comprising a rotation part configured to rotate the first adjustment optical device around a direction perpendicular to the light axis direction of the radiation light, wherein
the first adjustment optical device is rotatable around the direction perpendicular to the light axis direction of the radiation light, and
the control section controls the rotation part to rotate the first adjustment optical device around the direction perpendicular to the light axis direction of the radiation light in accordance with a movement amount of the first adjustment optical device by the second movement part.

8. The optical measurement device according to claim 1 further comprising:
a second adjustment optical device configured to collect the measurement light and emit the measurement light toward the probe;
a third movement part configured to move the second adjustment optical device in a light axis direction of the measurement light; and
a fourth movement part configured to move the second adjustment optical device on a plane perpendicular to the light axis direction of the measurement light, wherein,
when the probe emits the measurement light to the measurement target and receives the radiation light radiated from the measurement target, the control section controls the third movement part to move the second adjustment optical device in the light axis direction of the measurement light, and determines as a first spot diameter a diameter of a region where an intensity of the radiation light incident on the spectroscope is equal to a predetermined value,
the control section controls the fourth movement part to move the second adjustment optical device on a plane perpendicular to the light axis direction of the measurement light on the first spot diameter basis, and determines a position of the second adjustment optical device where the intensity of the radiation light incident on the spectroscope is greatest, and
the control section moves the second adjustment optical device on a diameter basis on the plane perpendicular to the light axis direction of the measurement light around a determined position of the second adjustment optical device, and determines a position of the second adjustment optical device where the intensity of the radiation light incident on the spectroscope is greatest, the diameter being smaller than the first spot diameter.

9. The optical measurement device according to claim 8, wherein the predetermined value is 1/e² of a peak of an intensity of the radiation light computed on a basis of a reflectance of the measurement target.

10. The optical measurement device according to claim 8, wherein the predetermined value is ½ of a peak of an intensity of the radiation light computed on a basis of a reflectance of the measurement target.

11. The optical measurement device according to claim 1 further comprising:
a third adjustment optical device configured to collect illumination light to be emitted to the measurement target from the probe, and emit the illumination light toward the probe;
a fifth movement part configured to move the third adjustment optical device in a light axis direction of the illumination light; and
a sixth movement part configured to move the third adjustment optical device on a plane perpendicular to the light axis direction of the illumination light, wherein,
when the probe emits the illumination light to the measurement target and receives the illumination light radiated from the measurement target, the third adjustment optical device collects the illumination light received by the probe and emits the illumination light toward the spectroscope,
when the probe emits the illumination light to the measurement target and receives the illumination light radiated from the measurement target, the control section controls the fifth movement part to move the third adjustment optical device in the light axis direction of the illumination light, and determines as a second spot diameter a diameter of a region where an intensity of the illumination light incident on the spectroscope is equal to a predetermined value,
the control section controls the sixth movement part to move the third adjustment optical device on the plane perpendicular to the light axis direction of the illumination light on the second spot diameter basis, and determines a position of the third adjustment optical device where the intensity of the illumination light incident on the spectroscope is greatest, and
the control section moves the third adjustment optical device on a diameter smaller than the second spot diameter basis, on the plane perpendicular to the light axis direction of the illumination light around a determined position of the third adjustment optical device, and determines a position of the third adjustment optical device where the intensity of the illumination light incident on the spectroscope is greatest.

12. The optical measurement device according to claim 11, wherein the predetermined value is 1/e² of a peak of an intensity of the illumination light radiated from the measurement target, the peak being computed on a basis of a reflectance of the measurement target.

13. The optical measurement device according to claim 11, wherein the predetermined value is ½ of a peak of an intensity of the illumination light radiated from the measurement target, the peak being computed on a basis of a reflectance of the measurement target.

14. A probe system comprising:
a probe configured to emit measurement light to a measurement target, and receive radiation light radiated from the measurement target; and
the optical measurement device according to claim 1.

15. The probe system according to claim 14, wherein the probe emits measurement light to a measurement target whose reflectance is known, and receives radiation light radiated from the measurement target.

16. The probe system according to claim 14, wherein
the probe includes a plurality of connecting terminals,
the probe and the optical measurement device are connected with each other through the plurality of connecting terminals,
the plurality of connecting terminals include a measurement optical fiber and a light reception fiber,
the optical measurement device includes a measurement light source unit and a light reception unit, and
the measurement optical fiber and the measurement light source unit face each other whereas the light reception fiber and the light reception unit face each other when the probe and the optical measurement device are connected to each other.
